(19) United States
(12) Patent Application Publication
(10) Pub. No.: US 2006/0229499 A1
(43) Pub. Date: Oct. 12, 2006

(54) Medical-technology valve device for suction and/or flushing lines of medical instruments

(76) Inventors: Peter Eisenkolb, Tuttlingen (DE); Andreas Efinger, Rietheim (DE)

Correspondence Address: ST. ONGE STEWARD JOHNSTON & REENS, LLC, 986 BEDFORD STREET, STAMFORD, CT, 06905-5619, US

(21) Appl. No.: 11/409,800

(22) Filed: Apr. 24, 2006

Related U.S. Application Data
(63) Continuation of application No. PCT/EP05/04388, filed on Apr. 23, 2005.

(30) Foreign Application Priority Data
Apr. 24, 2004 (DE) 10 2004 020 071.8

(51) Int. Cl. A61B 1/12

(52) U.S. Cl. 600/159; 600/156

(57) ABSTRACT

The invention relates to a medical-technology valve device for suction and/or flushing lines of medical instruments, particularly instruments for endoscopic surgery, having a valve housing equipped with a through bore-hole and having a valve body, movably positioned in the valve housing, by which valve body the through bore-hole can be locked and opened again, and where the valve body is configured as a piston that can be displaced essentially perpendicularly to the through bore-hole by means of a servo drive, so that a rotary movement of the servo drive can be transformed into an axial movement of the piston. To produce a medical-technology valve device that makes possible a reproducible adjustment of the flow volume that is to be regulated, while allowing a reduction of turbulence, it is proposed with the invention that the servo drive is configured as a hand lever that can rotate around the longitudinal axis of the piston and can be displaced to move the piston from the position that frees the through bore-hole into the position that closes the through bore-hole by an angle of 90 degrees with respect to the longitudinal axis of the piston.

### CROSS-REFERENCE TO RELATED APPLICATIONS

The present application is a continuation of pending International patent application PCT/EP2005/004388 filed on Apr. 23, 2005 which designates the United States and claims priority from German patent application 10 2004 020 071.8 filed on Apr. 24, 2004, the content of which is incorporated herein by reference.

### FIELD OF THE INVENTION

The invention relates to a medical-technology valve device for suction and/or flushing lines of medical instruments, particularly instruments for endoscopic surgery, having a valve housing equipped with a through bore-hole and having a valve body, movably positioned in the valve housing, by which valve body the through bore-hole can be locked and released again, and where the valve body is configured as a piston that can be displaced essentially perpendicularly to the through bore-hole by means of a servo drive, so that a rotary movement of the servo drive can be transformed into an axial movement of the piston.

### BACKGROUND OF THE INVENTION

In endoscopic surgery in particular, it is necessary to flush the operating area continually and to suction off the flushing liquid and blood from the operating area in order to ensure good illumination and visual conditions in the operating area and to make possible a clearly visible surgery. Suction and flushing as a rule are carried out by separate suction-flushing units with connected suction and/or flushing lines. These suction and/or flushing lines can be either fed into the operating area by separate accessways or else integrated into an operating tool.

To be able to operate the suction and/or flushing function in a deliberate and quantitatively controlled manner, the suction and/or flushing lines are provided with medical-technology valve devices, by which the suction and/or flushing lines can be opened and closed again.

The U.S. Pat. No. 4,794,913 A teaches a medical-technology valve device for suction and/or flushing lines of medical instruments, in particular instruments for endoscopic surgery, which is equipped with a rotatable hand lever. This familiar valve device serves to close off a flow channel leading from axial to radial. The diversion of the stream in this construction, however, causes severe turbulence, which prevents a reproducible adjustment of the volume flow that is to be regulated.

Along with additional faucets known in practice, with a through-bored stopcock, it is known from DE 691,22,314 T2, how to configured the valve devices as sliding valves. These known sliding valves have a valve body lockably positioned in the valve housing, so that the valve body is provided with a pass-through bore hole, which, when the valve is pressed, aligns flush with the through bore-hole in the valve housing and thus forms the open position of the valve device.

It is a disadvantage in valve devices configured as faucets and sliding valves that the regulation of the volume stream can be performed and reproduced only with difficulty. In addition, the known valve devices cause strong turbulence in the volume flow that is to be regulated.

On this basis, it is the aim of the invention to create a medical-technology valve device of the aforementioned type, which makes possible a reproducible adjustment of the volume flow that is to be regulated while reducing turbulence.

### SUMMARY OF THE INVENTION

This aim is fulfilled through the invention in a manner characterized by the fact that the servo drive is configured as a hand lever that can rotate around the longitudinal axis of the piston and can be displaced by a 90 degree angle with respect to the longitudinal axis of the piston in order to move the piston from the position releasing the through bore-hole into the position locking the through bore-hole.

Because of the configuration of the servo drive according to the invention, the valve device can be rapidly opened and closed because it requires only a slight motion of the servo drive in order to move the piston into the particular end position.

Because of the configuration of the valve body as a piston that can move into the through bore-hole of the valve housing, it becomes easy to reduce the flow volume in a reproducible manner. Because the piston does not have a through bore-hole, the displacement of the piston into the through bore-hole of the valve housing, or out of the bore-hole, causes a direct closing or opening of the connected flushing and/or suction channels. In addition, the piston according to the invention causes less turbulence in the flow volume that is to be regulated than the turbulence caused by valve devices known in the art.

According to a first practical embodiment of the invention, the servo drive and the piston are coupled to one another by means of a thread, which effects the transmission of the rotary motion of the servo drive into the axial motion of the piston.

According to a second practical embodiment of the invention, the servo drive and the piston are coupled to one another by means of a thread so that, in this embodiment, the servo drive is preferably configured as a hand lever that can rotate perpendicularly to the longitudinal axis of the piston.

In order to be able to regulate the flow volume by means of the valve device in a simply and reliably reproducible manner, it is proposed with the invention that a scale, which can indicate the position of the piston to the through bore-hole, is positioned on the valve housing and/or on the servo drive. In addition to, or as an alternative to, the scale, the servo drive can be configured so that it can be displaced in pre-established stepwise stops.

It is further proposed with the invention that the pitch of the thread, which transmits the rotary motion of the servo drive into the axial motion of the piston, is configured in such a way that the angle of rotation of the servo drive is proportional to the blocking or release of the through bore-hole, that is, proportional to the flow-through quantity, so that the thread preferably has a self-restricting action in order to prevent the through-flowing medium from affecting the valve position.

It is finally proposed with the invention that the motion of the piston can be directed by a blocking element in order to ensure that the rotary motion of the servo drive does not cause a rotation of the piston, but rather ensures the axial motion of the piston. This blocking element is advantageously configured as a rod that can be inserted into the valve housing and that extends into a longitudinal groove in the piston so that it blocks any turning of the piston.

Additional characteristics and advantages of the invention can be seen with reference to the related illustrations, in which an embodiment of a medical-technology valve device, according to the invention, for suction and/or flushing lines of medical instruments is depicted in exemplary manner.

### BRIEF DESCRIPTION OF THE DRAWINGS

FIG. 1 shows a side view of a medical-technology valve device according to the invention, in opened position.

FIG. 2 shows a section along the cut line II-II seen in FIG. 1.

FIG. 3 shows a side view according to FIG. 1 but depicting the valve device in partially closed position.

FIG. 4 shows a section along the cut line IV-IV seen in FIG. 3.

FIG. 5 shows a side view of the medical-technology valve device according to the invention, turned at an angle of 90 degrees with respect to the views in FIGS. 1 and 3.

FIG. 6 shows a section along the cut line VI-VI seen in FIG. 5.

### DETAILED DESCRIPTION OF THE INVENTION

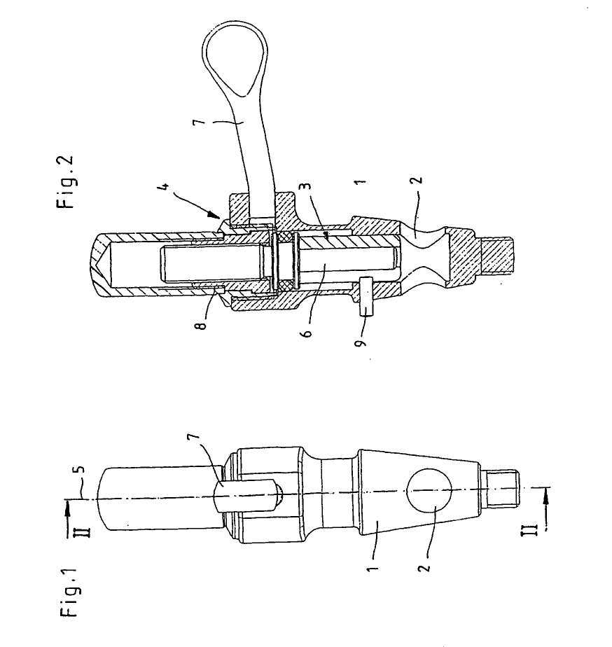

The medical-technology valve device illustrated in FIGS. 1 through 6 consists essentially of a valve housing 1 with a through bore-hole 2, onto which suction and/or flushing lines (not illustrated) of medical instruments can be connected, as well as a valve body 3 movably positioned in the valve housing 1.

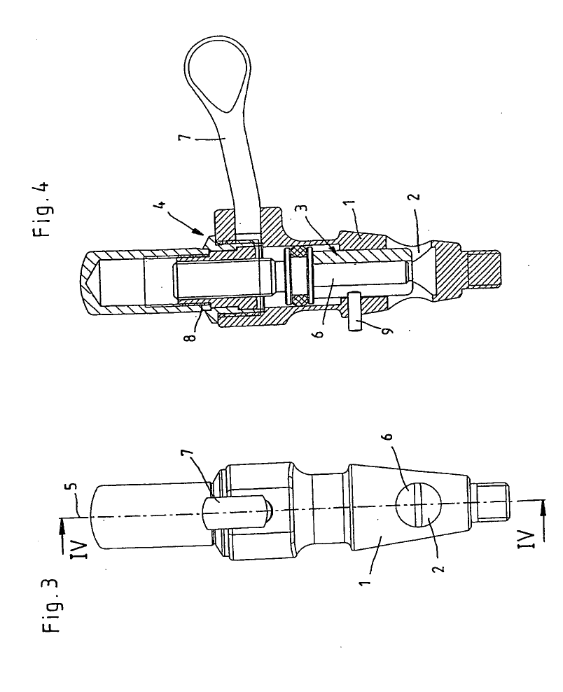

The valve body 3, as can be seen in FIGS. 1 through 4, is positioned in the valve housing 1 so that it can be displaced, by means of a servo drive 4, in the direction of the longitudinal axis 5 of the valve housing 1 between an open position (FIGS. 1 and 2) that releases the through bore-hole 2 and a closed position that closes off the through bore-hole. FIGS. 3 and 4 shows the valve body 3 in a half-opened or half-closed intermediate position.

As can be seen from the illustrations, the valve body 3 is configured as a closed piston 6, which has no through bore-hole of its own, so that every displacement of the piston 6 by the servo drive 4 signifies a direct closing or opening of the through bore-hole 2 and thus, in addition, a direct reduction or increase of the flow volume moving through the through bore-hole 2.

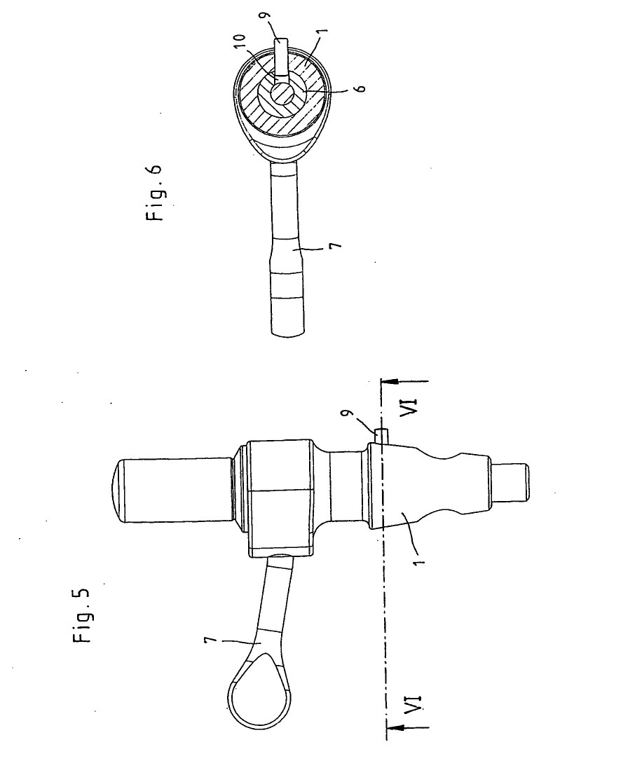

The servo drive 4 for activating the piston 6 is configured as a hand lever 7 that can rotate around the longitudinal axis 5 of the piston 6 and that is coupled with the piston 6 by means of a thread 8 in such a way that this thread converts the rotary motion of the hand lever 7 into the axial motion of the piston 6. To ensure that the rotation of the hand lever 7 does not cause a rotation of the piston 6 but causes only an up or down motion of the piston 6, a blocking element 9 is provided that is configured as a rod. As seen in particular in FIGS. 5 and 6, the blocking element 9, which is inserted radially into the valve housing 1, extends into a longitudinal groove 10 of the piston 6 and thus prevents rotation of the piston 6.

Because, contrary to the valve devices in the art, consisting of valve bodies, the valve body 3 in the form of a piston 6 has no through bore-hole of its own, every displacement of the valve body is in a direct proportion to the reduction or release of the flow volume that is to be regulated, so that this valve device makes possible a reproducible and easily controllable regulation of the flow volume streaming through the connected suction and/or flushing lines.

The reproducible controllability can be improved still further if a scale is positioned on the valve housing 1 and/or on the servo drive 4, which serves to indicate the position of the piston 6 to the through bore-hole 2. Likewise it is possible, in addition or alternatively, to configure the servo drive 4 in such a way that it can be displaced in pre-established stepwise stops.

It is also possible to configure the pitch of the thread 8 that converts the rotary motion of the servo drive into the axial motion of the piston 6 in such a way that the angle of rotation of the servo drive 4 is proportional to the blocking or opening of the through bore-hole 2, that is, proportional to the flow-through quantity, so that the thread 8 advantageously has a self-restricting effect, in order to prevent the through-flowing medium from influencing the valve position.

An additional advantage of the illustrated medical-technology valve device consists in the fact that the valve body 3 configured in this manner produces less turbulence, and/or more controllable turbulence, in opening and closing the through bore-hole 2 so that once again controllability of the amount is facilitated.

1. A medical-technology valve device for suction and/or flushing of medical instruments, in particular instruments for endoscopic surgery, having a valve housing provided with a through bore-hole as well as a valve body, movably positioned in the valve housing, by means of which valve body the through bore-hole can be closed and opened up again, whereby the valve body is configured by a piston that can be displaced essentially perpendicularly to the through bore-hole by means of a servo drive, so that a rotary motion of the servo drive can be converted into an axial motion of the piston, characterized in that the servo drive is configured as a hand lever that can rotate around the longitudinal axis of the piston and can be displaced to move the piston from the position that opens the through bore-hole into the position that closes the through bore-hole by a 90 degree angle with respect to the longitudinal axis of the piston.

2. A medical-technology valve device according to claim 1, characterized in that the servo drive and the piston are coupled to one another by means of a thread.

3. A medical-technology valve device according to claim 1, characterized in that the servo drive and the piston are coupled to one another by means of a gear.

4. A medical-technology valve device according to claim 3, characterized in that the servo drive is configured as a hand lever that can rotate perpendicularly to the longitudinal axis of the piston.

5. A medical-technology valve device according to claims 1, characterized in that on the valve housing and/or on the servo drive a scale is positioned that indicates the position of the piston with respect to the through bore-hole.

6. A medical-technology valve device according to claim 1, characterized in that the servo drive can be displaced in pre-established stepwise stops.

7. A medical-technology valve device according to claim 2, characterized in that the pitch of the thread is configured in such a way that the angle of rotation of the servo drive is porportional to the position of the piston to the through bore-hole.

8. A medical-technology valve device according to claim 7, characterized in that the thread is configured so that it is self-restricting.

9. A medical-technology valve device according to claim 1, characterized in that the motion of the piston can be controlled by a blocking element.

10. A medical-technology valve device according to claim 9, characterized in that the blocking element is configured as a rod that can be inserted into the valve housing.

11. A medical-technology valve device according to claim 9, characterized in that a longitudinal groove for the blocking element is configured in the piston.

12. A medical-technology valve device according to claim 1, characterized in that the angle of rotation of the servo drive can be restricted by a stop.
